(19) United States
(12) Patent Application Publication
(10) Pub. No.: US 2015/0011987 A1
(43) Pub. Date: Jan. 8, 2015

(54) BLOOD VESSEL INSERTION-TYPE TREATMENT DEVICE

(71) Applicant: TERUMO KABUSHIKI KAISHA, Shibuya-ku (JP)

(72) Inventors: Risato KOBAYASHI, Ashigarakami-gun (JP); Junichi KOBAYASHI, Ashigarakami-gun (JP); Ryota SUGIMOTO, Ashigarakami-gun (JP); Ichirou HIRAHARA, Ashigarakami-gun (JP)

(73) Assignee: TERUMO KABUSHIKI KAISHA, Shibuya-ku (JP)

(21) Appl. No.: 14/494,151

(22) Filed: Sep. 23, 2014

Related U.S. Application Data
(63) Continuation of application No. PCT/JP2013/001539, filed on Mar. 8, 2013.

(30) Foreign Application Priority Data
Mar. 23, 2012 (JP) 2012-067127

(51) Int. Cl. A61N 7/02

(52) U.S. Cl. CPC A61N 7/02
USPC 606/27

(57) ABSTRACT

A blood vessel insertion-type treatment device has a first ultrasonic transmitter and an insertion body. The first ultrasonic generator has a first ultrasonic transducer and a first actuator. The first ultrasonic transducer radiates cauterizing ultrasonic waves converging on a converging position. The first actuator 108 adjusts a direction of the converging position with respect to the first ultrasonic transducer. The insertion body has a longitudinal shape whose both ends have a proximal end and an insertion end. The first ultrasonic generator 106 is disposed near the insertion end in the insertion body.

### CROSS-REFERENCES TO RELATED APPLICATIONS

This application is a continuation of International Application No. PCT/JP2013/001539 filed on Mar. 8, 2013, and claims priority to Japanese Application No. 2012-067127 filed on Mar. 23, 2012, the entire content of both of which is incorporated herein by reference.

### TECHNICAL FIELD

The present invention generally relates to a blood vessel insertion-type treatment device. More specifically, the invention pertains to a blood vessel insertion-type treatment device which is configured to be inserted into a blood vessel to perform cauterization on biological tissues around the blood vessel through the inside of the blood vessel.

### BACKGROUND DISCUSSION

In recent years, it is understood that abnormal renal artery sympathetic nerve activity causes congestive heart failure, renal failure, hypertension, and other cardio-renal diseases. In addition, it is also known that these diseases are treated by removing a renal artery sympathetic nerve. In order to perform cauterization on the renal artery sympathetic nerve, a renal neuromodulation apparatus has been proposed which inserts an electrode into a renal artery and applies a pulse output electric field to the renal artery exchange nerve from the electrode. An example of this apparatus is disclosed in Japanese Application Publication No. 2008-515544.

In the cauterization of the renal artery sympathetic nerve which is performed by the renal neuromodulation apparatus disclosed in Japanese Application Publication No. 2008-515544 using the pulse output electric field, current density in a blood vessel intima increases to the maximum. For this reason, heat generated in the blood vessel intima increases to the maximum. Therefore, there is a possibility that the cauterization is performed on the entire vessel wall including the blood vessel intima. Consequently, side effects such as intimal thickening and thrombosis may occur.

### SUMMARY

A blood vessel insertion-type treatment device is disclosed which is configured to cauterize biological tissue around a blood vessel, such as the sympathetic nerve around a renal artery, while also suppressing damage to the blood vessel.

According to one aspect, a blood vessel insertion-type treatment device comprises: an elongated insertion body possessing an insertion end configured to be inserted into a blood vessel and a proximal end; and an ultrasonic generator axially movably positioned in the insertion body. The ultrasonic generator includes an ultrasonic transducer which radiates cauterizing ultrasonic waves converging on a converging position and an actuator which adjusts the ultrasonic transducer to change a location of the converging position at which converge the cauterizing ultrasonic waves radiated by the ultrasonic transducer.

The cauterizing ultrasonic waves converging on the converging position cauterize biological tissue on the converging position. Therefore, it is possible to suppress damage to blood vessels interposed between the ultrasonic generator and cauterizing target tissues. In addition, the actuator changes the direction from the ultrasonic transducer to the converging position. Therefore, without being limited to one specific point, it is possible to cauterize the biological tissue around the blood vessel even while an ultrasonic generator is used.

According to another aspect, a blood vessel insertion-type treatment device includes a sheath configured to be inserted into and moved along a blood vessel, wherein the sheath possesses an open distal end, an elongated insertion body positioned in the sheath and axially movable relative to the sheath to project a distal end portion of the elongated insertion body distally beyond the open distal end of the sheath, an ultrasonic transducer which radiates cauterizing ultrasonic waves that converge, wherein the ultrasonic transducer is located at the distal end portion of the elongated insertion body, and an actuator on which the ultrasonic transducer is mounted so that the ultrasonic transducer overlies the actuator and which is operable to adjust a location at which the cauterizing ultrasonic waves radiated by the ultrasonic transducer converge. The actuator is disposed on the elongated insertion body so that the actuator and the elongated insertion body move together as a unit.

In accordance with another aspect, a method of treating a treatment location comprises: inserting an elongated body into a blood vessel, with an ultrasonic transducer which radiates cauterizing ultrasonic waves that converge being located at a distal end portion of the elongated insertion body and being movable together with the elongated body as a unit; moving the elongated body along the blood vessel to position the distal end portion of the elongated body adjacent the treatment location; adjusting tilt of the ultrasonic transducer to change a converging position at which the cauterizing ultrasonic waves radiated by the ultrasonic transducer converge so that the converging position is at the treatment location; and radiating cauterizing ultrasonic waves at the treatment location to cauterize the treatment location.

### BRIEF DESCRIPTION OF DRAWINGS

FIG. 1 is a schematic illustration of a portion of a human body illustrating a manual technique for removing a renal artery sympathetic nerve using a blood vessel insertion-type treatment device according to a first embodiment representing one example of the blood vessel insertion-type treatment device disclosed here.

FIG. 2 is an enlarged view illustrating the vicinity of a renal artery into which a guiding catheter is inserted.

FIG. 3 is a longitudinal cross-sectional view near an insertion end of the blood vessel insertion-type treatment device according to the first embodiment.

FIG. 4 illustrates a case where a first ultrasonic transducer tilts along a first tilting plane by a first actuator.

FIG. 5 illustrates a case where a first ultrasonic transducer tilts along a second tilting plane by a first actuator.

FIG. 6 illustrates a case where an imaging ultrasonic transducer tilts along a third tilting plane by a second actuator.

FIG. 7 is a longitudinal cross-sectional view near an insertion end of a blood vessel insertion-type treatment device according to a second embodiment representing another example of the blood vessel insertion-type treatment device disclosed here.

FIG. 8 is a view illustrating a first modified example of an expandable member used in the blood vessel insertion-type treatment device.

FIG. 9 is a cross-sectional view taken along the section line IX-IX in FIG. 8.

FIG. 10 is a view illustrating a second modified example of an expandable member used in the blood vessel insertion-type treatment device.

FIG. 11 is a cross-sectional view taken along the section line XI-XI in FIG. 10.

FIG. 12 is a cross-sectional view of a blood vessel insertion-type treatment device inside a blood vessel taken along a direction perpendicular to a longitudinal direction, which illustrates a third modified example of an expandable member used in the blood vessel insertion-type treatment device.

FIG. 13 is a cross-sectional view of a blood vessel insertion-type treatment device inside a blood vessel taken along a direction perpendicular to a longitudinal direction, which illustrates a fourth modified example of an expandable member used in the blood vessel insertion-type treatment device.

### DETAILED DESCRIPTION

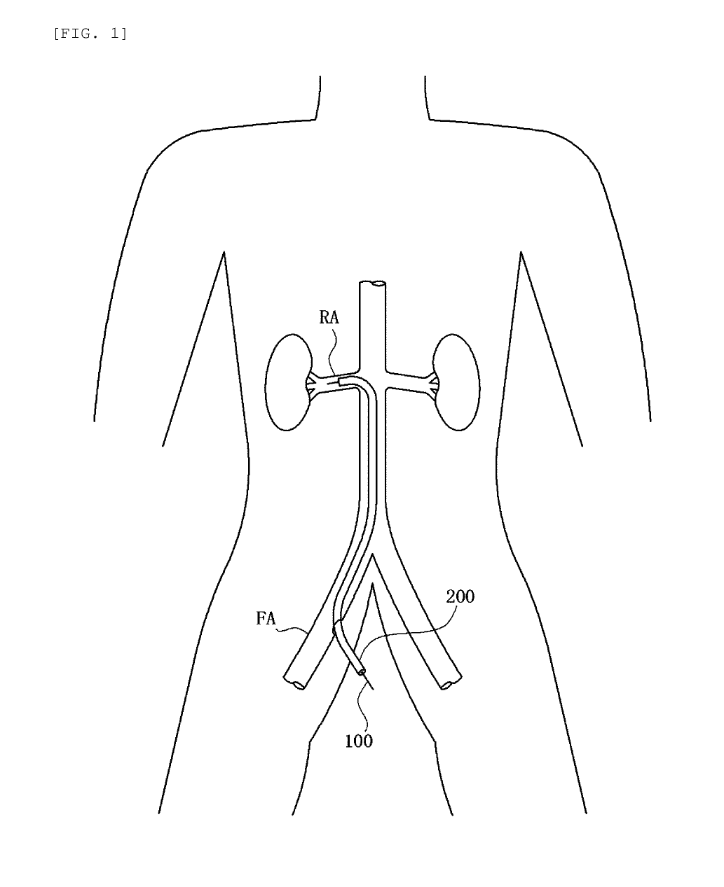

Hereinafter, embodiments of a blood vessel insertion-type treatment device representing examples of the blood vessel insertion-type treatment device disclosed here will be described with reference to the drawings. FIG. 1 schematically illustrates a manual technique to remove a renal artery sympathetic nerve using the blood vessel insertion-type treatment device according to a first embodiment of the blood vessel insertion-type treatment device.

Referring to FIG. 1, to apply a manual technique to remove the renal artery sympathetic nerve, a surgeon inserts a guiding catheter 200 into a femoral artery FA through a patient's thigh, and causes the distal end of the guiding catheter 200 to reach a renal artery RA. That is, after inserting the guiding catheter 200 into the patient's femoral artery FA, the surgeon advances the catheter so that the distal end of the guiding catheter 200 is positioned at the renal artery RA. A guide wire is used so that the guiding catheter 200 reaches the renal artery RA.

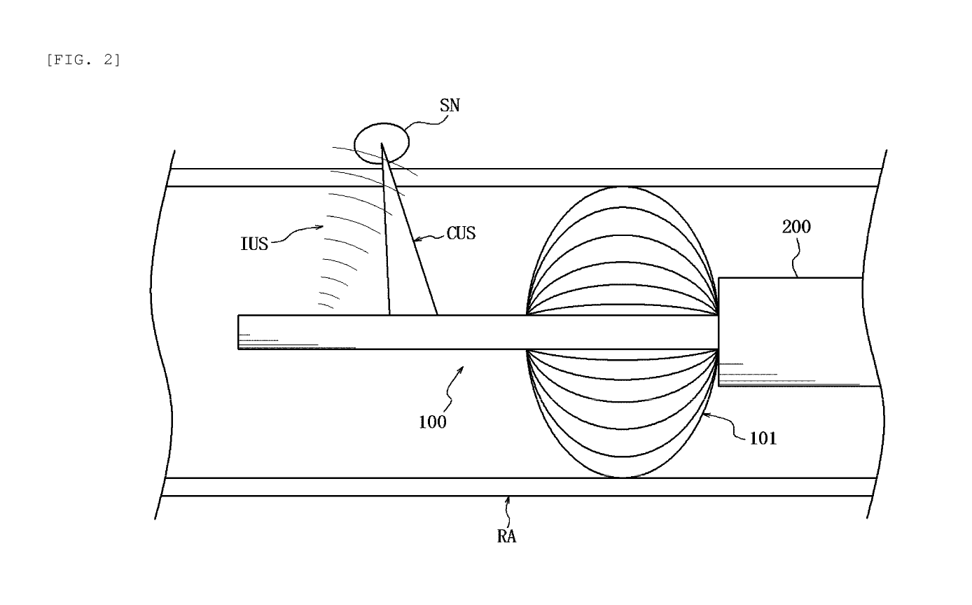

The guiding catheter 200 has a tubular shape, and medical examination and treatment devices can be inserted into the guiding catheter 200. A blood vessel insertion-type treatment device 100 possesses an overall elongated shape, has an insertion end and a proximal end, and is insertable into a lumen of the guiding catheter 200 through the insertion end. The surgeon inserts the blood vessel insertion-type treatment device 100 into the guiding catheter 200, and causes the insertion end to protrude from the guiding catheter 200 (refer to FIGS. 1 and 2). That is, the surgeon inserts the distal end of the blood vessel insertion-type treatment device 100 into the proximal end of the guiding catheter 200, and then advances the blood vessel insertion-type treatment device 100 along the guiding catheter so that the distal end portion of the blood vessel insertion-type treatment device 100 protrudes distally beyond the distal end of the guiding catheter 200 as shown in FIGS. 1 and 2. In a protruding state of the insertion end of the blood vessel insertion-type treatment device 100, an expandable member 101 disposed near the insertion end (distal end) of the blood vessel insertion-type treatment device 100 is outwardly expanded, thereby fixing the blood vessel insertion-type treatment device 100 in the renal artery RA.

As described below, the blood vessel insertion-type treatment device 100 has an imaging function and a cauterizing function. To fulfill the imaging function, the blood vessel insertion-type treatment device 100 is configured to radiate imaging ultrasonic waves (IUS in FIG. 2). The imaging ultrasonic waves are identified as IUS in FIG. 2. The surgeon causes the inserted blood vessel insertion-type treatment device 100 to fulfill the imaging function, thereby causing the blood vessel insertion-type treatment device 100 to acquire an image around the renal artery through the inside of the renal artery RA.

Based on the acquired image, the surgeon determines a sympathetic nerve SN to be cauterized, and adjusts the position of the blood vessel insertion-type treatment device 100 so that cauterizing ultrasonic waves are radiated to the determined sympathetic nerve SN. The cauterizing ultrasonic waves are identified as CUS in FIG. 2. After adjusting the position, the surgeon causes the blood vessel insertion-type treatment device 100 to fulfill the cauterizing function, and cauterizes a desired sympathetic nerve.

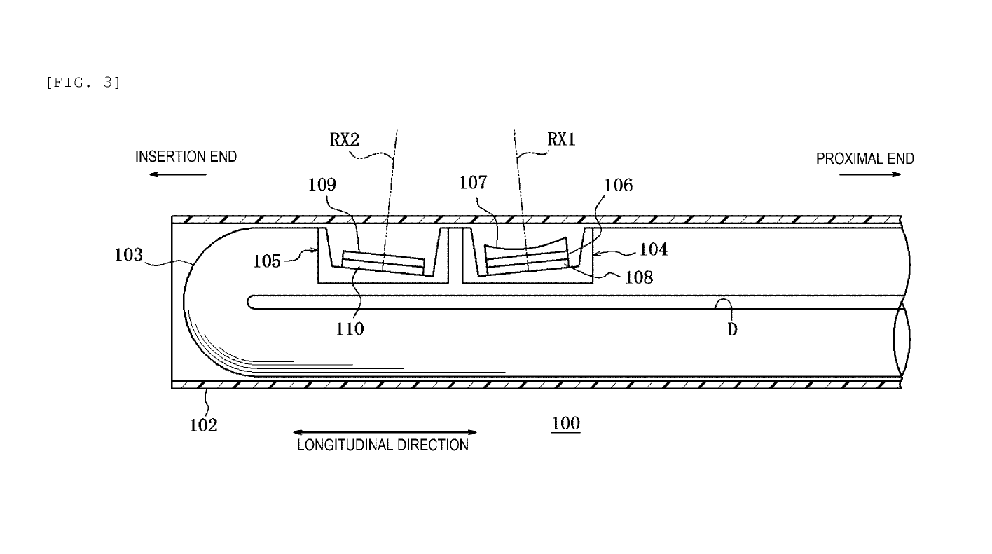

Next, a configuration of the blood vessel insertion-type treatment device 100 will be described with reference to FIG. 3. The blood vessel insertion-type treatment device 100 is configured to include a sheath 102, an insertion body 103, a first ultrasonic generator 104, an image acquisition unit 105, and an expandable member 101 (shown in FIG. 2).

The sheath 102 possesses a tubular shape and is a member having acoustic characteristics and flexibility. A distal end portion on the insertion end side of the sheath 102 is open. In addition, when the sheath 102 starts to be used, the sheath 102 is internally filled, from the proximal end, with a medium having acoustic transmission characteristics or properties. A tongue piece (not illustrated) extending to an inner surface is formed on the proximal side of the sheath 102.

The insertion body 103 is a flexible member extending from the proximal end of the sheath 102 to the insertion end (distal end) of the sheath 102. In a state where the insertion end or distal end of the insertion body 103 is positioned at the insertion end or distal end of the sheath 102, the proximal end of the insertion body 103 protrudes from the proximal end of the sheath 102. The insertion body 103 is thus longer than the sheath 102.

The outer diameter of the insertion body 103 is narrower (smaller) than the inner diameter of the sheath 102, and the insertion body 103 is freely displaceable or axially movable inside the sheath 102 in the longitudinal direction. A longitudinally extending groove portion (groove) D is formed in the insertion body 103. The tongue piece of the sheath 102 engages (is positioned in) the groove portion D to prevent pivotal rotation of the insertion body 103 inside the sheath 102 about the longitudinal direction.

The first ultrasonic generator 104 is disposed near the insertion end or distal end of the insertion body 103. The first ultrasonic generator 104 is axially spaced from the image acquisition unit 105 along the longitudinal or axial extent of the insertion body 103. A recessed portion (recess) is formed near the insertion end of the insertion body 103, and the first ultrasonic generator 104 is embedded or positioned in the recessed portion. The first ultrasonic generator 104 has a single unit of a first ultrasonic transducer 106 (the first ultrasonic transducer 106 in this embodiment consists of a single ultrasonic transducer), an acoustic lens 107, and a first actuator 108.

The first ultrasonic transducer 106 possesses a flat plate shape, and radiates cauterizing ultrasonic waves CUS, having a frequency suitable for cauterization, from a plate surface. Depending on the frequency, a distance for transmitting the ultrasonic waves and a calorific value in a converging position of the ultrasonic waves are determined. Therefore, the frequency of the cauterizing ultrasonic waves CUS is predetermined, based on an approximate interval from the inside of the renal artery RA to the renal artery sympathetic nerve SN and the calorific value required for the cauterization of the sympathetic nerve SN.

A signal line, extending from the first ultrasonic transducer 106 to the proximal end of the insertion body 103, is connected to a cauterization control unit. The cauterization control unit supplies a drive signal to the first ultrasonic transducer 106 to generate the cauterizing ultrasonic waves CUS at the above-described frequency.

The acoustic lens 107 is disposed on a surface of the first ultrasonic transducer 106. The acoustic lens 107 causes the ultrasonic waves to converge at the converging position away from the acoustic lens 107 by a predetermined distance, thereby maximizing heat energy near the converging position. The acoustic lens 107 is configured to have a predetermined focal length, based on the approximate distance from the inside of the renal artery to the renal artery sympathetic nerve.

The first actuator 108 can cause a plate surface of the first ultrasonic transducer 106 on which the acoustic lens 107 is located to tilt from (deviate from) a first reference axis RX1. The first reference axis RX1 is normal (perpendicular) to the plate surface of the first ultrasonic transducer 106 in a state where the first actuator 108 is not driven or not operated.

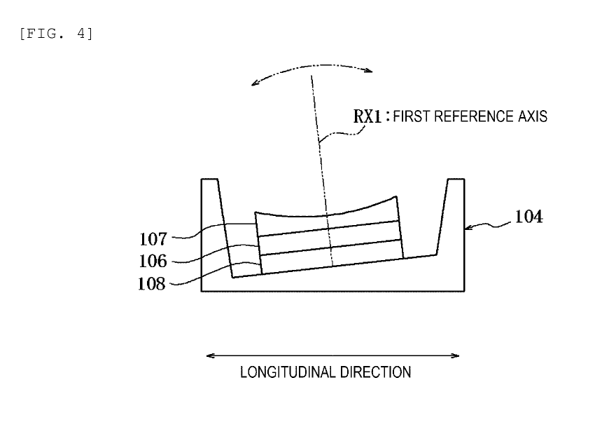

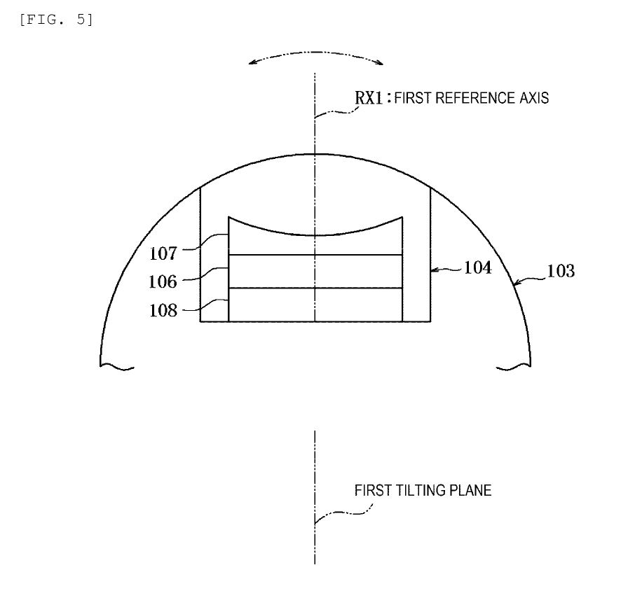

The first actuator 108 is configured to cause the first ultrasonic transducer 106 to tilt from the first reference axis RX1 in a direction along a first tilting plane (plane of the paper in FIGS. 3 and 4) which passes through the first reference axis RX1 and which is parallel to the longitudinal direction. In addition, the first actuator 108 is configured to cause the first ultrasonic transducer 106 to tilt from the first reference axis RX1 in a direction along a second tilting plane (plane of the paper in FIG. 5) which passes through the first reference axis RX1 and which is perpendicular to the first tilting plane.

A signal line, extending from the first actuator 108 to the proximal end of the insertion body 103, is connected to the cauterization control unit. The cauterization control unit supplies the first actuator 108 with a drive signal for causing the first ultrasonic transducer 106 to tilt along the first tilting plane and the second tilting plane. The first actuator 108 thus tilts or adjusts the position of the first ultrasonic transducer 106 relative to the elongated body 103, and the first actuator 108 performs this adjustment independent of any movement (rotational movement or axial movement) of the elongated body 103.

As illustrated in FIG. 3, the image acquisition unit 105 is disposed on the insertion end or distal end of the insertion body 103, at a position distal of the first ultrasonic generator 104. A recessed portion (recess) is located near the insertion end of the insertion body 103, and the image acquisition unit 105 is embedded or positioned in the recessed portion. The image acquisition unit 105 includes an imaging ultrasonic transducer 109 and a second actuator 110.

The imaging ultrasonic transducer 109 possesses a flat plate shape and generates, from the plate surface, imaging ultrasonic waves IUS suitable for acquisition of an image. In addition, the imaging ultrasonic transducer 109 generates a pixel signal corresponding to the reflected waves of the imaging ultrasonic waves IUS. The resolution of the reflected waves of the ultrasonic waves changes depending on the frequency of the ultrasonic waves. Based on the resolution required for confirmation and medical examination of the position of a specific sympathetic nerve, the frequency of the imaging ultrasonic waves IUS is predetermined.

A signal line, extending from the imaging ultrasonic transducer 109 to the proximal end of the insertion body 103, is connected to an imaging control unit. The imaging control unit supplies the imaging ultrasonic transducer 109 with a drive signal for generating the imaging ultrasonic waves IUS at the above-described frequency. In addition, the imaging control unit receives a pixel signal generated by the imaging ultrasonic transducer 109.

The imaging ultrasonic transducer 109 is configured to create an image of tissue, and can also observe a temperature change when the cauterizing ultrasonic waves are radiated and a status change in a state of the tissue. Reflection of the ultrasonic waves occurs at a boundary where acoustic impedance represented by the product of the density of a medium and sound speed of the medium varies. The acoustic impedance varies in such a manner that the density, the sound speed, or hardness of the tissue varies in response to the heating of the tissues. In this manner, a signal of the ultrasonic waves reflected on the tissues varies, and accordingly, it is possible to diagnose a cauterized state of the tissue.

The second actuator 110 is configured to cause the plate surface of the imaging ultrasonic transducer 109 to tilt from a second reference axis RX2. The second reference axis RX2 is normal (perpendicular) to the plate surface of the imaging ultrasonic transducer 109 in a state where the second actuator 110 is not driven or is not operated. In addition, the second reference axis RX2 is included in the first tilting plane (plane of the paper in FIG. 3), and tilts to or toward the first ultrasonic generator 104 side.

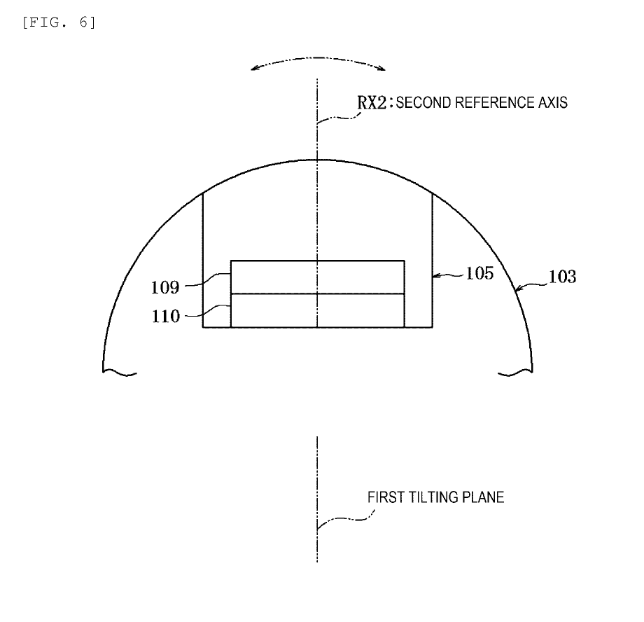

The second actuator 110 is configured to cause the imaging ultrasonic transducer 109 to tilt from the second reference axis RX2 in a direction along the first tilting plane (plane of the paper in FIGS. 3 and 4). In addition, the second actuator 110 is configured to cause the imaging ultrasonic transducer 109 to tilt from the second reference axis RX2 in a direction along a third tilting plane (plane of the paper in FIG. 6) which passes through the second reference axis RX2 and which is perpendicular to the first tilting plane. The second actuator 110 thus tilts or adjusts the position of the imaging ultrasonic transducer 109 relative to the elongated body 103, and the second actuator 110 performs this adjustment independent of any movement (rotation movement or axial movement) of the elongated body 103.

A signal line, extending from the second actuator 110 to the proximal end of the insertion body 103, is connected to the imaging control unit. The image control unit supplies the second actuator 110 with a drive signal for causing the imaging ultrasonic transducer 109 to tilt along the first tilting plane and the third tilting plane.

The imaging control unit estimates multiple locations to which the imaging ultrasonic waves are radiated, based on a drive signal transmitted to the second actuator 110 or information for generating the drive signal. The imaging control unit creates an image, based on an image signal and a position of the estimated radiation locations of the imaging ultrasonic waves.

The expandable member 101 is disposed on the sheath 102. A wire configuring the expandable member 101 is bent outward from the blood vessel insertion-type treatment device 100, and the wire presses against the inner wall of the blood vessel. In this manner, the blood vessel insertion-type treatment device 100 can be fixed in the blood vessel.

According to the blood vessel insertion-type treatment device 100 of the first embodiment having the above-described configuration, it is possible to maximize heat energy at the converging position of the cauterizing ultrasonic waves. It is thus possible to cauterize the biological tissues distributed in a range from the inside of the blood vessel to the outside of the blood vessel, while also suppressing damage to the blood vessel interposed between the biological tissues.

In addition, with the blood vessel insertion-type treatment device 100 of the first embodiment, it is possible to change the facing direction of the converging position from the first ultrasonic transducer 106 using the first actuator 108.

In the cauterization of the biological tissue using the ultrasonic transducer, the ultrasonic waves are caused to converge on a focus (focus region). Consequently, a region where the cauterization is possible is only in the vicinity of the focus. Therefore, in the present embodiment, the first actuator 108 is used to change the direction from the first ultrasonic transducer 106 to the converging position. In this manner, it is possible to cauterize the biological tissue distributed at various positions near the insertion end of the sheath 102.

In addition, according to the blood vessel insertion-type treatment device 100 of the first embodiment, the image acquisition unit 105 is disposed near the first ultrasonic generator 104. Therefore, it is relatively easy to confirm the biological tissue to be cauterized, and to confirm the cauterized state.

In particular, the image acquisition unit 105 uses the second actuator 110 to change the posture of the imaging ultrasonic transducer 109 which radiates the imaging ultrasonic waves. In this manner, it is possible to scan the biological tissue around the blood vessel by using the imaging ultrasonic waves.

In addition, according to the blood vessel insertion-type treatment device 100 of the first embodiment, it is possible to temporarily fix the position of the insertion end of the blood vessel insertion-type treatment device 100 in the blood vessel using the expandable member 101. It is possible to reduce a blur in a reproduced image by fixing the blood vessel insertion-type treatment device 100. In addition, it is possible to reduce a blur occurring at the radiation position of the cauterizing ultrasonic waves CUS. Also, because an expandable member 101 is used, it is possible to ensure the blood flow. Accordingly, it is possible to prevent overheating of an inner wall portion of the blood vessel to which the cauterizing ultrasonic waves CUS are radiated, while the blood vessel insertion-type treatment device 100 is fixed in position in the blood vessel.

Next, a blood vessel insertion-type treatment device according to a second embodiment will be described. The second embodiment differs from the first embodiment in that the image acquisition unit is integrated with the first ultrasonic generator. The following description of the second embodiment focuses primarily on differences between this second embodiment and the first embodiment described above and illustrated in FIGS. 1-6. Features common to both embodiments are identified by common reference numerals and a detailed description of such features is not repeated.

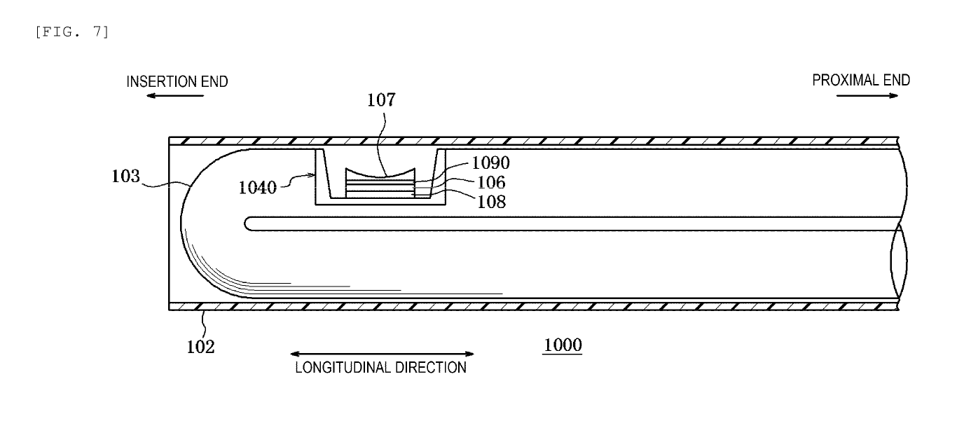

As illustrated in FIG. 7, a blood vessel insertion-type treatment device 1000 according to the second embodiment includes the sheath 102, the insertion body 103, a first ultrasonic generator 1040, and the expandable member 101 (shown in FIG. 2). In the second embodiment, unlike the first embodiment, the image acquisition unit is not separately provided. The configuration and function of the sheath 102, the insertion body (first torque transmission body) 103, and the expandable member 101 are the same as the configuration and function of such of those features in the first embodiment.

A first ultrasonic generator 1040 is disposed near the insertion end or distal end of the insertion body 103. A recessed portion (recess) is positioned near the insertion end of the insertion body 103, and the first ultrasonic generator 1040 is embedded or positioned in the recessed portion. The first ultrasonic generator 1040 has a single unit of the first ultrasonic transducer 106 (the first ultrasonic transducer 106 in this embodiment consists of a single ultrasonic transducer), the acoustic lens 107, the first actuator 108, and an imaging ultrasonic transducer 1090. As shown in FIG. 7, the acoustic lens 107 overlies the imaging ultrasonic transducer 1090, the first ultrasonic transducer 106 and the actuator 108, the imaging ultrasonic transducer 1090 overlies the ultrasonic transducer 106 and the actuator 108, and the ultrasonic transducer 106 overlies the actuator 108.

The configuration and function of the first ultrasonic transducer 106, the acoustic lens 107, and the first actuator 108 are the same as those in the first embodiment. Therefore, similar to the first embodiment, it is possible to radiate the cauterizing ultrasonic waves CUS to converge on the converging position away from the first ultrasonic generator 1040 by a predetermined distance. In addition, similar to the first embodiment, the first actuator 108 is configured to tilt the first ultrasonic transducer 106 in the direction along the first tilting plane and the second tilting plane.

In this second embodiment, the imaging ultrasonic transducer 1090 is disposed between the first ultrasonic transducer 106 and the acoustic lens 107. For example, the imaging ultrasonic transducer 1090 is formed of a piezoelectric film sheet, and can generate the imaging ultrasonic waves IUS. In addition, the imaging ultrasonic transducer 1090 generates a pixel signal corresponding to the reflected waves of the imaging ultrasonic waves IUS.

The imaging ultrasonic transducer 1090 together with the first ultrasonic transducer 106 can also tilt in response to the drive or operation of the first actuator 108, in the direction along the first tilting plane and the second tilting plane.

According to the blood vessel insertion-type treatment device 1000 of the second embodiment which has the above-described configuration, it is also possible to maximize heat energy at the converging position of the cauterizing ultrasonic waves. Therefore, whereas it is possible to cauterize the biological tissues distributed in a range from the inside of the blood vessel to the outside of the blood vessel, it is also possible to suppress damage to the blood vessel interposed between the biological tissues.

In addition, according to the blood vessel insertion-type treatment device 1000 of the second embodiment, it is also possible to cauterize the biological tissues distributed at various positions near the insertion end of the sheath 102, by driving or operating the first actuator 108. It is also possible to scan the biological tissues around the blood vessel by using the imaging ultrasonic waves. In addition, according to the blood vessel insertion-type treatment device 1000 of the second embodiment, it is possible to temporarily fix the vicinity of the insertion end of the blood vessel insertion-type treatment device 1000 in the blood vessel using the expandable member 01. In addition, since the expandable member 101 is used, it is possible to ensure the blood flow. Accordingly, it is possible to prevent overheating of the inner wall portion of the blood vessel to which the cauterizing ultrasonic waves CUS are radiated, while the blood vessel insertion-type treatment device 100 is fixed into the blood vessel.

Set forth above is a detailed description of examples of the blood vessel insertion-type treatment device disclosed here. However, it should be noted that those skilled in the art can easily perform various modifications and corrections based on the present disclosure. Therefore, all these modifications and corrections are intended to be included within the scope of the present invention.

For example, the blood vessel insertion-type treatment device 100, 1000 of the first and second embodiments includes the expandable member 101. However, a configuration may be employed in which the blood vessel insertion-type treatment device 100, 1000 is temporarily fixed in the blood vessel using other balloons.

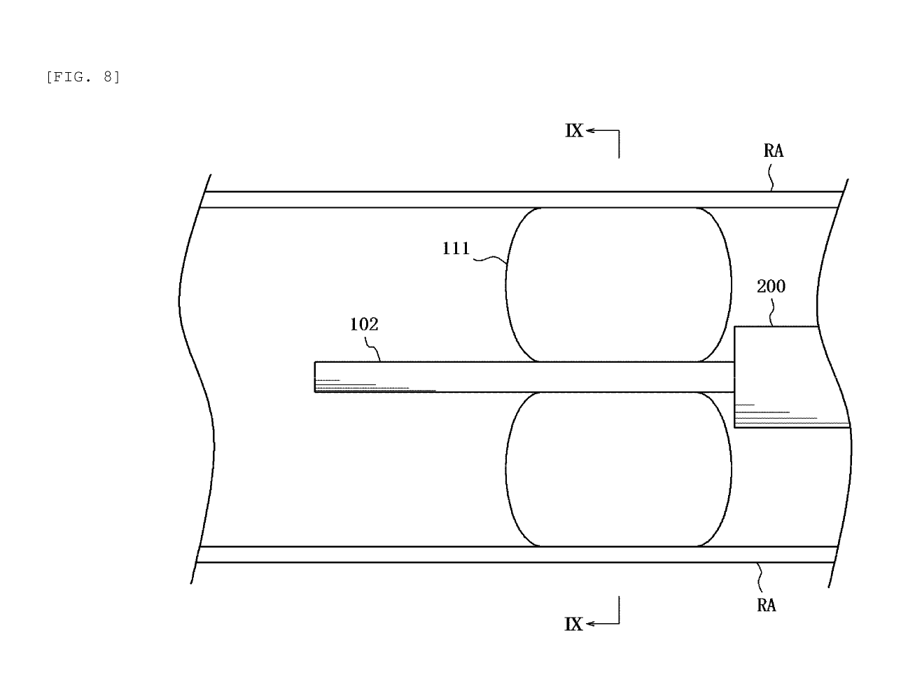

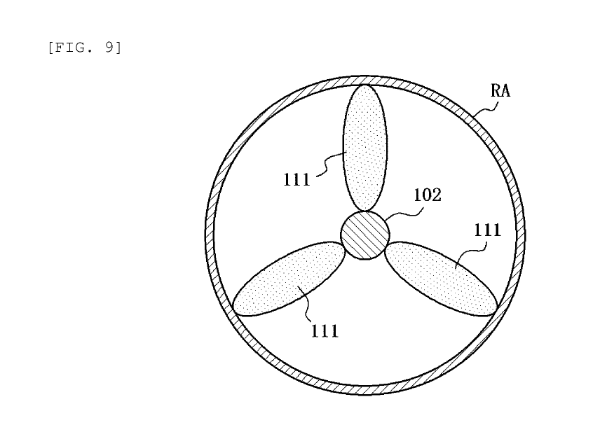

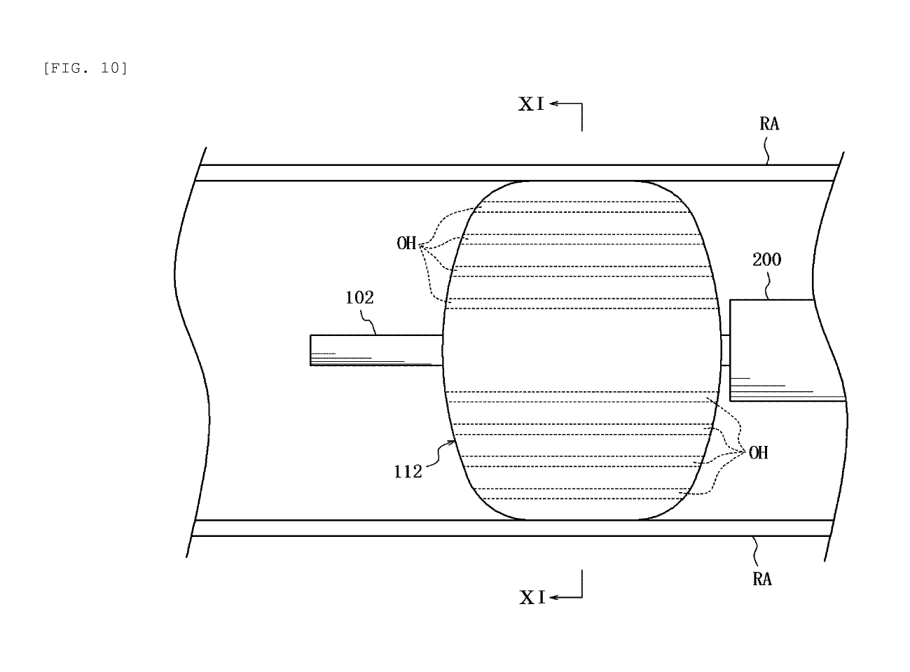

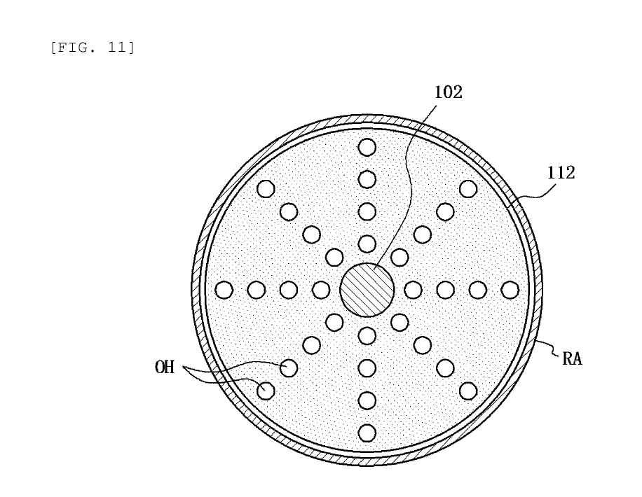

In particular, it is preferable to use a balloon for preventing overheating of the inner wall of the blood vessel. For example, as illustrated in FIGS. 8 and 9, it is possible to obtain an overheating prevention effect which is the same as that of the expandable member 101 by employing a configuration having multiple balloons 111 which can expand around the center of the sheath 102 in different directions. In addition, for example, as illustrated in FIGS. 10 and 11, it is also possible to obtain the overheating prevention effect which is the same as that of the expandable member 101 by employing a configuration having a balloon 112 which can expand to the entire periphery around the center of the sheath 102, and which has hole portions OH penetrating in the longitudinal direction.

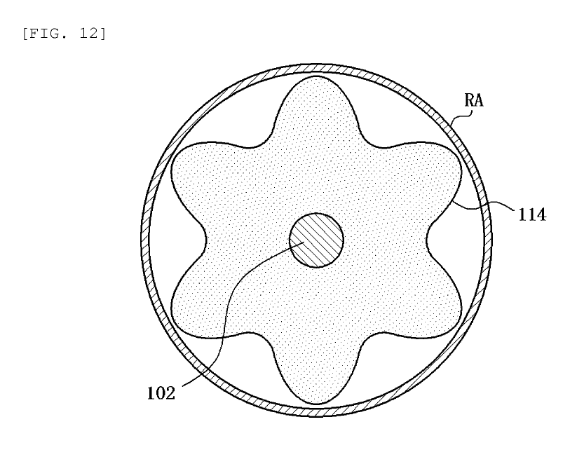

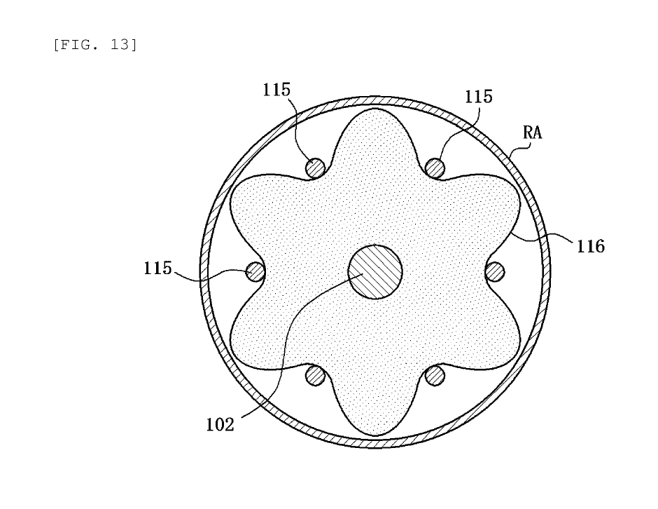

In addition, for example, as illustrated in FIG. 12, it is also possible to obtain the overheating prevention effect which is the same as that of the expandable member 101 by employing a configuration having a balloon 114 formed so that a cross section along a plane perpendicular to the longitudinal direction has a star shape. In addition, for example, as illustrated in FIG. 13, it is also possible to obtain the overheating prevention effect which is the same as that of the expandable member 101 by employing a configuration in which multiple wires 115 are used so as to partially expand a balloon 116. That is, the wires 115 inhibit the portions of the balloon at which the wires 115 are located from expanding as much as the remaining portions of the balloon where the wires are not located.

Alternatively, it is preferable to use a perfusion balloon and a cryo-balloon in which the inner wall of the blood vessel can be cooled by a refrigerant. In the cauterization using the ultrasonic waves, it is possible to maximize the heating energy at the focus (focal region/point). However, blood vessel walls including the inner wall of the blood vessel through which the ultrasonic waves are propagated prior to convergence may also be heated by the ultrasonic waves. Therefore, it is possible to further reduce a possibility of damage which may occur on the inner wall of the blood vessel, by using the cooling-type balloon.

In addition, in the first embodiment, the first actuator 108 is configured to cause the first ultrasonic transducer 106 to tilt along both the first tilting plane and the second tilting plane. However, the first actuator 108 may be configured to cause the first ultrasonic transducer 106 to tilt along at least any one tilting plane. In addition, the second actuator 110 is configured to cause the imaging ultrasonic transducer 109 to tilt along both the first tilting plane and the third tilting plane. However, the second actuator 110 may be configured to cause the imaging ultrasonic transducer 109 to tilt along at least any one tilting plane.

The first actuator 108 and the second actuator 110 may be configured to cause the first ultrasonic transducer 106 and the imaging ultrasonic transducer 110 to tilt along only the first tilting plane. According to this configuration, it is also possible to cauterize the biological tissues distributed along the circumferential direction of the blood vessel, and to acquire the image of the biological tissues, by rotating the blood vessel insertion-type treatment device 100 about the longitudinal direction. In addition, according to this configuration, without disposing a tongue piece of the sheath 102 in a groove portion D of the insertion body 103, it is also possible to cauterize the biological tissues distributed along the circumferential direction of the blood vessel, and to acquire the image of the biological tissues, by pivotally rotating the insertion body 103 inside the sheath 102 in the longitudinal direction.

The first actuator 108 and the second actuator 110 may be configured to cause the first ultrasonic transducer 106 and the imaging ultrasonic transducer 110 to be respectively tilted along only the second tilting plane and the third tilting plane. According to this configuration, it is also possible to cauterize the biological tissue distributed along the longitudinal direction of the blood vessel, and to acquire the image of the biological tissue by displacing the insertion body 103 inside the sheath 102 in the longitudinal direction.

In addition, in the first embodiment, the image acquisition unit 105 is configured to acquire the image by using the ultrasonic waves, but may be configured to acquire the image, based on optical information such as TD-OCT and HUD-OCT.

In the second embodiment, the imaging ultrasonic transducer 1090 is configured to be interposed between the first ultrasonic transducer 106 and the acoustic lens 107, but may be configured to be interposed between the first ultrasonic transducer 106 and the first actuator 108.

The detailed description above describes features and aspects of embodiments of a blood vessel insertion-type treatment device and manner of use/operation of a blood vessel insertion-type treatment device. The invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A blood vessel insertion-type treatment device comprising:
a sheath configured to be inserted into and moved along a blood vessel, the sheath possessing an open distal end;
an elongated insertion body positioned in the sheath and axially movable relative to the sheath to project a distal end portion of the elongated insertion body distally beyond the open distal end of the sheath;
an ultrasonic transducer which radiates cauterizing ultrasonic waves that converge, the ultrasonic transducer being located at the distal end portion of the elongated insertion body;
an actuator on which the ultrasonic transducer is mounted so that the ultrasonic transducer overlies the actuator and which is operable to adjust a location at which the cauterizing ultrasonic waves radiated by the ultrasonic transducer converge; and
the actuator being disposed on the elongated insertion body so that the actuator and the elongated insertion body move together as a unit.

2. The blood vessel insertion-type treatment device according to claim 1, wherein an outer surface of the elongated insertion body includes a recess, the actuator and the ultrasonic transducer being positioned in the recess.

3. The blood vessel insertion-type treatment device according to claim 1, wherein the ultrasonic transducer is a first ultrasonic transducer, and further comprising a second ultrasonic transducer configured to detect imaging ultrasonic waves radiated by the ultrasonic generator and reflected waves of the imaging ultrasonic waves, the second ultrasonic transducer being disposed on the elongated insertion body in axially spaced apart relation to the first ultrasonic transducer so that the second ultrasonic transducer and the elongated insertion body move together as a unit.

4. The blood vessel insertion-type treatment device according to claim 3, wherein the actuator is a first actuator, and further comprising a second actuator on which the second ultrasonic transducer is mounted so that the second ultrasonic transducer overlies the second actuator and which is operable to adjust a posture of the second ultrasonic transducer.

5. The blood vessel insertion-type treatment device according to claim 1, further comprising an outwardly expandable balloon disposed on the insertion body which fixes a position of the elongated body in the vessel when the balloon is outwardly expanded into contact with an inner surface of the vessel.

6. The blood vessel insertion-type treatment device according to claim 5, wherein the balloon is positioned proximally of the ultrasonic transducer.

7. The blood vessel insertion-type treatment device according to claim 1, wherein the ultrasonic transducer is a first ultrasonic transducer, and further comprising a second ultrasonic transducer configured to detect imaging ultrasonic waves radiated by the ultrasonic generator and reflected waves of the imaging ultrasonic waves, the second ultrasonic transducer overlying the first ultrasonic transducer.

8. The blood vessel insertion-type treatment device according to claim 1, wherein the ultrasonic transducer is a first ultrasonic transducer, and further comprising a second ultrasonic transducer configured to detect imaging ultrasonic waves radiated by the ultrasonic generator and reflected waves of the imaging ultrasonic waves, the second ultrasonic transducer overlying the first ultrasonic transducer and the first actuator, and the first actuator being configured to adjust a posture of the second ultrasonic transducer.

9. A blood vessel insertion-type treatment device comprising:
an elongated insertion body possessing an insertion end configured to be inserted into a blood vessel and a proximal end; and
an ultrasonic generator axially movably positioned in the insertion body, the ultrasonic generator including an ultrasonic transducer which radiates cauterizing ultrasonic waves converging on a converging position and an actuator which adjusts the ultrasonic transducer to change a location of the converging position at which converge the cauterizing ultrasonic waves radiated by the ultrasonic transducer,

10. The blood vessel insertion-type treatment device according to claim 9, wherein the ultrasonic transducer is a first ultrasonic transducer and the actuator is a first actuator, and further comprising:
an image acquisition unit disposed on the insertion body adjacent the first ultrasonic generator of the insertion body, the image acquisition unit including a second ultrasonic transducer configured to detect imaging ultrasonic waves radiated by the ultrasonic generator and reflected waves of the imaging ultrasonic waves; and
a second actuator which adjusts a posture of the second ultrasonic transducer.

11. The blood vessel insertion-type treatment device according to claim 9, further comprising:
a tubular sheath covering the insertion body and the ultrasonic generator; and
a balloon disposed on the insertion body near an end portion of the insertion body on an insertion end side of the insertion body, the insertion body being outwardly expandable around the sheath.

12. The blood vessel insertion-type treatment device according to claim 11, wherein the balloon is a cooling balloon that prevents overheating of a portion which comes into contact with the balloon when the balloon expands.

13. The blood vessel insertion-type treatment device according to claim 9, wherein the ultrasonic transducer is a first ultrasonic transducer, and further comprising an image acquisition unit that includes a second ultrasonic transducer configured to detect imaging ultrasonic waves radiated by the ultrasonic generator and reflected waves of the imaging ultrasonic waves, the second ultrasonic transducer being axially spaced apart from the first ultrasonic transducer along an axial extent of the elongated insertion body.

14. The blood vessel insertion-type treatment device according to claim 13, the second ultrasonic transducer being mounted on a second actuator which adjusts a posture of the second ultrasonic transducer.

15. The blood vessel insertion-type treatment device according to claim 9, wherein the ultrasonic transducer is a first ultrasonic transducer, and further comprising an image acquisition unit that includes a second ultrasonic transducer configured to detect imaging ultrasonic waves radiated by the ultrasonic generator and reflected waves of the imaging ultrasonic waves, the second ultrasonic transducer overlying the first ultrasonic transducer.

16. The blood vessel insertion-type treatment device according to claim 9, wherein the ultrasonic transducer is a first ultrasonic transducer, and further comprising an image acquisition unit that includes a second ultrasonic transducer configured to detect imaging ultrasonic waves radiated by the ultrasonic generator and reflected waves of the imaging ultrasonic waves, the second ultrasonic transducer overlying the first ultrasonic transducer and the first actuator, and the first actuator being configured to adjust a posture of the second ultrasonic transducer.

17. The blood vessel insertion-type treatment device according to claim 9, wherein the ultrasonic generator also includes an acoustic lens overlying both the ultrasonic transducer and the actuator.

18. A method of treating a treatment location comprising:
inserting an elongated body into a blood vessel, with an ultrasonic transducer which radiates cauterizing ultrasonic waves that converge being located at a distal end portion of the elongated insertion body and being movable together with the elongated body as a unit;
moving the elongated body along the blood vessel to position the distal end portion of the elongated body adjacent the treatment location;
adjusting tilt of the ultrasonic transducer to change a converging position at which the cauterizing ultrasonic waves radiated by the ultrasonic transducer converge so that the converging position is at the treatment location; and
radiating cauterizing ultrasonic waves at the treatment location to cauterize the treatment location.

19. The method according to claim 18, wherein the inserting of the elongated body into the blood vessel includes inserting the elongated body into a renal artery, and wherein the radiating of the cauterizing ultrasonic waves at the treatment location includes radiating cauterizing ultrasonic waves at a sympathetic nerve to cauterize the sympathetic nerve.

20. The method according to claim 18, wherein the ultrasonic transducer is a first ultrasonic transducer, the method further comprising acquiring an image around the blood vessel by radiating imaging ultrasonic waves and detecting reflected waves of the imaging ultrasonic waves using a second ultrasonic transducer disposed on the elongated body.
